(19) United States
(12) Patent Application Publication
(10) Pub. No.: US 2024/0050724 A1
(43) Pub. Date: Feb. 15, 2024

(54) TOPICAL WORKOUT MUSCLE RECOVERY COMPOSITION AND ITS METHOD OF APPLICATION

(71) Applicant: Annadurai Dharmarajan, Morrisville, NC (US)

(72) Inventor: Annadurai Dharmarajan, Morrisville, NC (US)

(21) Appl. No.: 18/088,131

(22) Filed: Dec. 23, 2022

Related U.S. Application Data
(63) Continuation-in-part of application No. 17/885,163, filed on Aug. 10, 2022.

(51) Int. Cl. A61M 35/00; A61K 31/045; A61K 31/125; A61K 31/618; A61K 31/522; A61K 31/455; A61K 33/14; A61K 33/00; A61K 31/4045; A61K 31/198; A61K 31/167

(52) U.S. Cl. CPC A61M 35/10; A61K 31/045; A61K 31/125; A61K 31/618; A61K 31/522; A61K 31/455; A61K 33/14; A61K 33/00; A61K 31/4045; A61K 31/198; A61K 31/167; A61M 2205/6081; A61M 2205/6063

(57) ABSTRACT

The disclosure relates to a topical workout muscle recovery composition that includes a pain reliever, water for hydration, an amino acid and inorganic salts, method of use along with a adhesive bandage pod (Appod™). Muscle recovery and the pain often present after a workout are traditionally constituted treated by sitting into an ice bath and/or by taking nutritional supplements along with minerals as a drink or an energy bar by mouth or oral route. When taken orally, the supplements and minerals are provided to whole body, but during a workout, certain muscles will be used and go through fatigue and pain. The invention relates to topical workout muscle recovery compositions that includes a hands-free applicator along with Appod™ on to the skin that can be applied to specific part of the body and provide pain relief. In some embodiments, the amino acid and mineral supplement can be required for muscle recovery.

### CROSS-REFERENCE TO RELATED APPLICATIONS

This application is a continuation-in-part of U.S. patent application Ser. No. 17/885,163, filed Aug. 10 2022, which is hereby incorporated by reference in its entirety.

### TECHNICAL FIELD

The presently disclosed subject matter is generally directed to a topical workout muscle recovery composition and topical adhesive bandage pod (referred to as an “Appod™”), and to a convenient method of making and using the disclosed system. Muscle recovery and the pain often present after a workout are traditionally treated by sitting in an ice bath and/or by taking nutritional supplements along with minerals as a drink or an energy bar by mouth or oral route. When taken orally, the supplements and minerals are provided to the whole body, but during a workout, certain muscles will be used and go through fatigue and pain. The invention relates to topical workout muscle recovery compositions that include a hands-free applicator along with Appod™ on the skin that can be applied to specific part of the body and provide pain relief. In some embodiments, the amino acid and mineral supplement can be required for muscle recovery.

### BACKGROUND

There are a number of products that purport to provide an individual with muscle recovery benefits when consumed after or during workout. These products are often packaged as oral supplement foods in powder form or as drinks and are consumed by individuals wanting to quickly recover physical energy (e.g., in particular situations such as after a workout or sports activities and to prevent cramps). For example, when individuals go to a gym and/or are involved in physical activity as a workout, they may workout longer and consume the products to train or compete.

Research suggests that when we exercise, the fuel we use results in an increase in our core temperature, which in turn causes us to sweat. It is essential to rehydrate when we sweat, as dehydration during exercise can lead to dizziness, muscle cramps, and microtrauma and tears in the muscle fibers affected, which causes the pain and soreness after exercise/workout. Immersion in an ice bath is considered one of the ways to accomplish muscle recovery. However, ice baths may decrease gains in strength and muscle growth. A 2015 study in the Journal of Physiology showed decreased long-term gains in muscle mass and strength, which is in line with a 2014 study in the Journal of Strength & Conditioning Research that showed decreases in strength using cold immersion. Potential side effects of ice bath immersion include hypothermia, nerve damage, and pain. Another approach that is widely followed is the use of nutritional supplements taken as liquids, as a powder dispersed in water, or as an energy bar. However, these products may include undesirable amounts sugar for the whole body, and nothing in these products will alleviate pain on the individual or affected muscles. For example, the inclusion of high amounts of salts and amino acids may provide for the entire muscles of the body. However, only a few muscles are typically involved in the athletic activity, and these supplements will not relieve the pain associated with the specific muscle workout. Further, the supplements may fail to provide sufficient physical recovery of specific muscles. Moreover, consumers continually desire unique and healthy formulations that can be used to recover specific muscles that are worked during the physical activity. Accordingly, there is a need for improved topical workout muscle recovery compositions that provide consumers with benefits such as (but not limited to) increased relief from muscle pain and improved and quicker recovery after a workout.

### BRIEF DESCRIPTION OF THE FIGURES

FIG. 1 is a top plan view of an adhesive bandage pod (Appod™) in accordance with some embodiments of the presently disclosed subject matter.

FIG. 2 is a bottom plan view of an adhesive bandage pod (Appod™) in accordance with some embodiments of the presently disclosed subject matter.

FIG. 3 is a perspective view of an applicator comprising disinfecting liquid comprising a sponge tip applicator.

### SUMMARY

In some embodiments, the presently disclosed subject matter includes a topical (applied directly to a skin of the body) workout muscle recovery composition that includes a pain reliever in an amount from about 0.05% to about 50%, an inorganic salt in an amount from about 0.05% to about 5.0% (w/w), water from 0.1% to 90% and at least one amino acid in an amount of about 0.01% to 20%, based on the total weight of the composition. Thus, the composition can comprise about 0.05-50 weight percent of a pain reliever (e.g., at least/no more than about 0.05, 0.1, 0.5, 1, 5, 10, 15, 20, 25, 30, 35, 40, 45, or 50 weight percent), based on the total weight of the composition. The composition can further include about 0.05-5 weight percent of an inorganic salt (e.g., at least/no more than about 0.05, 0.1, 1, 1.5, 2, 2.5, 3, 3.5, 4, 4.5, or 5 weight percent), based on the total weight of the composition. The composition can include water in an amount of from about 0.1-90 weight percent (e.g., at least/no more than about 0.1, 1, 5, 10, 15, 20, 25, 30, 35, 40, 45, 50, 55, 60, 65, 70, 75, 80, 85, or 90 weight percent), based on the total weight of the composition. The composition can include at least one amino acid in an amount of from about 0.01-20 weight percent (e.g., at least/no more than about 0.01, 0.1, 1, 5, 10, 15, or 20 weight percent), based on the total weight of the composition.

In some embodiments, the present invention includes a topical workout muscle recovery composition comprising pain reliever, water, an inorganic salt derivative, at least one amino acid derivative, at least one vitamin, and caffeine. Consumption of said composition provides a user with at least one benefit such as (but not limited to) pain relief, increased rate of muscle recovery, increased continuity of workout, stress relief, and/or anxiety relief.

In some embodiments, the present invention also relates to a topical workout muscle recovery composition that includes a pain reliever, an inorganic salt derivative, at least one vitamin, collagen, caffeine, and at least one amino acid.

The present invention also relates to a device or adhesive bandage pod or application pod, (referred to as Appod™) that holds the liquid or semiliquid on the skin in a specific area for an extended period of time without soiling the clothes or the skin of the user (human or animal).

In some embodiments, the presently disclosed subject matter is directed to a topical workout muscle recovery composition comprising a pain reliever; water; a nonsteroidal anti-inflammatory drug derivative selected from the group comprising diclofenac; ibuprofen; piroxicam; ketorolac; a local anesthetic drug derivative selected from the group comprising lidocaine, benzocaine; a rubefacient/counter irritant drug derivative selected from the group comprising methyl salicylate, trolamine salicylate, nicotinate esters, capsaicin derived from chili pepper, Capsicum minimum, menthol, minoxidil, camphor and thurfyl nicotinate; and naturally occurring plant derivatives selected from the group comprising Eucalyptus, clove, Arnica montana, Boswellia carterii, Calendula officinalis and Vamellia dinensis; and combinations thereof; at least one inorganic salt derived from Sodium, Magnesium, potassium, copper, strontium, zinc, or combinations thereof; an amino acid or a dipeptide and its salt selected from at least one member selected from the group comprising leucine, isoleucine, valine, glutamine, creatine, carnitine, bxetaine, beta-Alanine, taurine, and combinations thereof; a natural supplement selected from the group comprising melatonin, caffeine, L-theanine, ashwagandha, Ginseng, biotin, keratin, gamma ammino butyric acid, vitamin A, vitamin D, vitamin C, vitamin B complex, vitamin B12, and coenzyme Q10 (CoQ10), or combinations thereof, and at least one preservative. The composition is in liquid to semisolid in nature or consistency in an airless bottle.

In some embodiments, the presently disclosed subject matter is directed to a topical workout muscle recovery composition comprising: a pain reliever; Water; a nonsteroidal anti-inflammatory drug derivative selected from the group comprising diclofenac; ibuprofen; piroxicam; ketorolac; a local anesthetic drug derivative selected from lidocaine, Benzocaine; a rubefacient/counter irritant drug derivative selected from methyl salicylate or trolamine salicylate; nicotinate esters; capsaicin, derived from chili pepper; Capsicum minimum; menthol; minoxidil; camphor, thurfyl nicotinate; and naturally occurring plant derivatives selected from Eucalyptus, clove, Arnica Montana, Boswellia carterii, Calendula Officinalis and Camellia Sinensis; and combinations thereof; at least one member selected from amino acid or a dipeptides and its salts selected from the group containing Lucine, Isoleucine, valine, Glutamine, creatine, carnitine, bxetaine, beta-Alanine and taurine and combinations thereof, at least one natural supplement selected from melatonin, caffeine, L-theanine, ashwagandha, Ginseng, biotin, keratin, gamma ammino butyric acid, vitamin A, vitamin D, vitamin C, vitamin B complex, vitamin B12, and coenzyme Q10 (CoQ10); at least one preservative. The composition is in liquid to semisolid in nature or consistency in an airless bottle.

In some embodiments, the presently disclosed subject matter is directed to a topical workout muscle recovery composition water comprising: pain reliever; a nonsteroidal anti-inflammatory drug derivative selected from the group comprising diclofenac; ibuprofen; piroxicam; ketorolac; a local anesthetic drug derivative selected from the group lidocaine, benzocaine; a rubefacient/counter irritants drug derivative selected from the group methyl salicylate, trolamine salicylate; Nicotinate esters; Capsaicin derived from chili pepper; Capsicum minimum; Menthol; Minoxidil; camphor; Thurfyl nicotinate; and naturally occurring plant derivatives selected from Eucalyptus, clove, Arnica Montana, Boswellia carterii, Calendula Officinalis and Camellia Sinensis; and combinations thereof, at least one inorganic salt derived from Sodium, Magnesium, Potassium, copper, strontium, or zinc; at least one natural supplement selected from Melatonin, caffeine, L-Theanine, Ashwagandha, Ginseng, Biotin, Keratin, gamma ammino butyric acid, Vitamin A, vitamin D, vitamin C, vitamin B complex, vitamin B12, and Coenzyme Q10 (CoQ10); at least one preservative. The composition is in liquid to semisolid in nature or consistency in an airless bottle.

In some embodiments, the presently disclosed subject matter is directed to a topical workout muscle recovery composition water comprising: pain reliever; a nonsteroidal anti-inflammatory drug derivative selected from diclofenac; ibuprofen; piroxicam; ketorolac; a local anesthetic drug derivative selected from the group lidocaine, benzocaine; a rubefacient/counter irritants drug derivative selected from the group methyl salicylate and trolamine salicylate; Nicotinate esters; Capsaicin derived from chili pepper; Capsicum minimum; Menthol; Minoxidil; camphor, Thurfyl nicotinate; and naturally occurring plant derivatives selected from Eucalyptus, clove, Arnica Montana, Boswellia carterii, Calendula Officinalis and Camellia Sinensis; and combinations thereof; at least one inorganic salt derived from Sodium, Magnesium, Potassium, copper, strontium, or zinc; at least one member selected from amino acid or a dipeptides and its salts selected from the group containing Leucine, Isoleucine, valine, Glutamine, creatine, carnitine, bxetaine, beta-Alanine and taurine and combinations thereof. The composition is in liquid to semisolid in nature or consistency in an airless bottle.

In some embodiments, the composition is a liquid, is in a completely dissolved state and “ready to act form” and provides consistent effect as a solution or nanosized or micronized suspension, gel, or an emulsion with a viscosity of 2000 cP or less; and wherein the composition further comprises caffeine and collogen protein.

In some embodiments, the composition includes about 1% to 90% by weight of water and a skin permeation enhancer; wherein the composition is mixed with one or more polymers to provide a sustainable action for longer period; and wherein the composition can be applied using an THADS applicator in airless container or an Adhesive bandage pod.

In some embodiments, the adhesive bandage pod is configured to be applied on the skin of human or animals, and wherein the adhesive bandage pod holds the liquid or semi-liquid in place, and wherein the adhesive bandage pod is configured to prevent the soiling of cloths and provide hands-free application of a topical product or products.

In some embodiments, the adhesive bandage pod is configured to be applied to the skin, wherein the topical solution/product is added into the adhesive bandage pod for local or systemic applications, wherein the drug product is not adhered to adhesive.

In some embodiments, the adhesive bandage pod is configured to hold the topical composition in place and prevent any reduction or loss of the composition from any external contact of the treatment via rubbing of a hand or absorption of clothing.

In some embodiments, the adhesive bandage pod is configured to provide rapid onset and supply a constant rate of supply of topical products in an enclosed area of skin for an extended period and wherein the topical product can be refilled if needed.

In some embodiments, the adhesive bandage pod is configured to administer more than one topical product simultaneously in a same treatment area of skin for multimodal treatment.

In some embodiments, the adhesive bandage pod comprises an adhesive, an occlusive or non-occlusive polymer top layer, and a non-woven or woven fabric to hold or to spread the topical product, and a release liner.

In some embodiments, the adhesive bandage pod comprises a predetermined closed or a refillable chamber or holder for the topical product that can have predetermined quantities of topical product, wherein the topical product is released from the chamber or holder at a predetermined rate to an application area, wherein the adhesive bandage pod (Appod™) optimally comprises non-woven or woven fabric.

In some embodiments, said amino acid is glutamine or its derivatives in an amount from about 0.01% to about 20 weight percent, wherein the composition optionally further includes L-Analy-L Glutamine.

In some embodiments, the composition comprises about 0.05 mL-5 ml of water per pound of muscle and wherein the preservative is present in an amount from about 0.015% to about 2 weight % and is from sodium benzoate, potassium sorbate, benzoic acid, sorbic acid, levulinic acid, anisic acid, methylparaben, ethylparaben, propylparaben, butylparaben, isobutylparaben, methylisothiazolinone, imadozolidinyl urea, diazolidinyl urea, phenoxyethanol, caprylyl glycol, EDTA, and combinations thereof.

In some embodiments, said inorganic salt is present in an amount from about 0.1% to about 200% of daily volume.

In some embodiments, said caffeine is present in an amount from about 0.1% to about 40 weight % of the composition.

In some embodiments, the adhesive bandage pod comprises a frame having an upper surface, a lower surface, an outer boundary, and an inner boundary, wherein said inner boundary defines an interior opening configured to receive a topical dosage product selected from a solution suspension, emulsion ointment, cream, or topical powder.

In some embodiments, the adhesive bandage pod comprises an adhesive disposed on said lower surface of said frame; and a release liner removably coupled to said adhesive, wherein the adhesive in the said lower surface frame is continuous or discontinuous, and wherein the adhesive is positioned on all sides or on less than all sides of the frame.

In some embodiments, said amino acid is present in an amount of about 0.1-18 weight percent, and wherein the amino acid is selected from one or more of Glutamine, Alanine, Creatine, leucine, isoleucine, valine, or an amino acid derivative.

In some embodiments, the adhesive bandage pod comprises an outer layer material with an opening to add the topical product, a non-woven fabric layer or layers to spread or hold the topical products, wherein said outer layer material is transparent or non-transparent, waterproof, flexible, permeable to water vapor, and resistant to penetration by microorganisms.

In some embodiments, the composition is not bound in an adhesive within the wherein the adhesive bandage pod but is dispersed or spread free in an area defined by said interior opening of said frame and very close to the skin.

In some embodiments, said release liner of the adhesive bandage pod comprises multiple parts each removably coupled to said adhesive on the lower surface and also on said upper surface.

In some embodiments, at least two of said multiple parts of said adhesive bandage pod release liner overlap and comprise tabs for grasping and removing said release liner.

In some embodiments, said tabs are at least one of color coded or numbered to indicate a recommended order of removal.

In some embodiments, the adhesive bandage pod has a frame having an upper surface, a lower surface, an outer boundary which contains adhesive and an inner boundary which contains a silicone dome, wherein in between inner boundary and outer boundary lies an intermediate layer that comprises non-woven fabric that absorbs the liquid and holds and delivers the product to the skin.

### DETAILED DESCRIPTION

It is to be understood that the disclosed embodiments are merely exemplary, and details disclosed herein are not to be interpreted as limiting, but merely as a representative basis for teaching one skilled in the art.

The disclosure relates to a stable, topical workout muscle recovery composition. The term “topical” refers to the application of the disclosed composition at or immediately beneath the point of application. The terms “topically”, “topical administration” and “topically applying” are used interchangeably to refer to delivering the disclosed composition onto one or more surfaces of a tissue or cell, including epithelial surfaces (e.g., a user's skin). Topical administration generally provides a local rather than a systemic effect.

The term “muscle recovery” refers to the structural and/or functional repair of muscle tissue (e.g., muscle cells, muscle fibers, sarcomers). Muscle damage is the mechanical disruption of muscle fibers, its membrane or the surrounding connective tissue or tendons. Thus, a “workout recovery composition” can function to aid in muscle recovery. In some embodiments, a workout recovery composition can reduce the time required for muscle recovery.

The term “composition” refers to a product comprising specified ingredients as provided herein in specified or effective amounts. The composition may be in liquid and/or semi solid (e.g., properties partly of that of a solid and partly that of a liquid) form. The composition may be provided in any mass or volume. For example, the composition may be provided as a semi solid item in the form of gel, creams, ointment, emulsion, suspension, or, rub-on or roll-on or similar products. The composition may also be provided as a liquid such as a “spray”, roll-on, or extended topical product like transdermal products. In its various forms, the composition is stable as a topical workout muscle recovery composition for at least one year to five years.

In some embodiments, the presently disclosed subject matter includes a method and/or a device and/or adhesive bandage pod to hold or house the readily active liquids intended for topical application.

The use of the disclosed composition provides certain benefits. For example, the composition may eliminate, reduce, and/or prevent pain in the muscles. The composition may hydrate the specific muscles and provide minerals that can significantly increase the power of performance of the muscles, significantly increase workout recuperation, increase recovery from muscle fatigue, exhibit a faster or expedited recovery period after a workout, increase the ability to hydrate and replenish fluids lost during the muscle workout, enhance muscle recovery, provide energy, provide attention, relieve stress, enhance sleep, improve the quality of episodic memory, and/or increase the speed of memory and self-related alertness. The noted benefits can provide a perceived feeling of high performance and endurance.

The performance and physiological effects of the disclosed composition can be sustained for an extended period of time with Appod™. The effect of the composition on performance and muscle recovery is greater than would be observed than when ingesting an equivalent amount of any if the ingredients of the disclosed composition alone. For example, the quantity of camphor, methyl salicylate and menthol, L-glutamine, sodium and magnesium salts required topically on the site of application on the working muscle is significantly less than the quantity of the ingredient when consumed orally. In one embodiment, the composition may provide synergistic benefits compared to the effects of an equivalent amount of ingredients taken alone. In other words, the ingredients of the disclosed composition act synergistically to produce benefits to a consumer that exceed the benefits achieved when the ingredients are individually taken by oral route.

As used herein, the term “derivative” includes, but is not limited to, precursors, metabolites, structurally-similar compounds and analogs of a particular substance.

As used herein, the term “metabolites of a substance” include, but are not limited to, molecules that are produced in vivo by transformation of that substance.

As used herein, the term “structurally similar-compounds” include, but are not limited to, molecules that are structurally similar to the identified substance but possess at least one structural difference and are functionally similar.

As used herein, the term “analogs” are defined to include, but are not limited to, molecules that are chemically distinct from an identified substance but that exert the same biological activity.

The disclosed muscle recovery composition includes at least one inorganic salt (e.g., sodium chloride) and at least one amino acid (e.g., glutamine) derivative including precursors, structurally-similar compounds, analogs, and/or metabolites of these substances. The term “inorganic salt” generally refers to a compound comprising a metal cation and an anion. Suitable inorganic salts can include (but are not limited to) sodium salts (e.g., sodium chloride), potassium salts (e.g., potassium chloride, potassium sulfate), magnesium salts (e.g., magnesium chloride, magnesium phosphate), calcium salts (e.g., calcium chloride, calcium carbonate), aluminum salts (e.g., aluminum chloride), zirconium salts, zinc salts (e.g., zinc chloride), hydrochloric acid, sulfuric acid, nitric acid, carbonic acid, phosphoric acid, ammonium salts (e.g., ammonium sulfate, ammonium phosphate, ammonium chloride), or combinations thereof.

The disclosed composition may also include vitamins, amino acids, taurine, creatinine, caffeine, melatonin, pain relivers (e.g., menthol, methyl salicylate, camphor), choline, folate, and preservatives, including precursors, structurally-similar compounds, analogs and/or metabolites of these substances.

The term “vitamin” refers to trace organic substances that are required in the diet of a human or animal. Suitable vitamins can include (but are not limited to) thiamin, riboflavin, nicotinic acid, pantothenic acid, pyridoxine, biotin, folic acid, vitamin B12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ10), pyridoxal phosphate, biocytin, tetrahydrofolic acid, coenzyme B12, lipoyllysine, 11-cis-retinal, and 1,25-dihydroxycholecalciferol. The term vitamin(s) also includes choline, camitine, and alpha, beta, and gamma carotenes. Thus, the disclosed composition may include vitamin B6. Vitamin B6, in its various forms, is involved in enzymatic reactions involved in amino acid metabolism and the metabolism of one-carbon units, carbohydrates, and lipids. Vitamin B6 is also required for the biosynthesis of neurotransmitters. Active individuals typically have higher glycogen stores and need adequate vitamin B6 to assure that the energy stored in glycogen can be quickly released when it is needed for energy during exercise. Vitamin B6 is also involved in gluconeogenesis, glycogenolysis and immune function. It serves as a coenzyme for a key enzyme involved in the mobilization of single-carbon functional groups (one-carbon metabolism) including reactions that are involved in the synthesis of nucleic acids. The effect of vitamin B6 deficiency on the function of the immune system may be partly related to its role in one-carbon metabolism.

In some embodiments, the vitamin includes vitamin B9 (folate). Folate is required for DNA and RNA synthesis. Folate is required to make normal red blood cells and to prevent anemia. Folate is also essential for the metabolism of homocysteine, helping to maintain normal levels of this amino acid. A deficiency of folate, vitamin B12, or vitamin B6 may increase blood levels of homocysteine, and folate supplementation has been shown to decrease homocysteine levels and to improve endothelial cell function. At least one study has linked low dietary folate intake with an increased risk of coronary events. Some evidence associates low blood levels of folate with a greater risk of cancer, possibly through increased DNA damage that may lead to cancer. In one embodiment, the folic acid is present in an amount from about 0.00001% to about 0.09% (or about 0.0000001 g/ml to about 0.0002 g/ml). Thus, the composition can comprise at least about (or no more than about) 0.00001, 0.00005, 0.0001, 0.0005, 0.001, 0.005, 0.01, 0.05, 0.09 weight percent based on the total weight of the composition). The composition may provide from about 10% to about 100% (e.g., at least/no more than about 10, 20, 30, 40, 50, 60, 70, 80, 90, or 100%) of the recommended daily allowance of folic acid.

In some embodiments, the composition includes vitamin B12. Vitamin B12 exists in several forms that include the mineral cobalt, e.g., methylcobalamin and 5-deoxyadenosylcobalamin. Vitamin B12 is required for proper red blood cell formation, neurological function, and DNA synthesis. In one embodiment, vitamin B12 is present in the disclosed composition in an amount from about 0.0001% to about 0.003% (e.g., at least/no more than about 0.0001, 0.0005, 0.001, 0.002, 0.003 weight percent based on the total weigh of the composition). The composition may provide about 50% to about 10000% of the recommended daily allowance of vitamin B12 (e.g., at least/no more than about 50, 75, 100, 500, 1000, 2000, 3000, 4000, 5000, 6000, 7000, 8000, 9000, or 10000 percent).

The muscle recovery composition may include niacin (vitamin B3) and/or niacinamide. In one embodiment, niacinamide is present in an amount from about 0.00001% to about 0.5% (e.g., at least/no more than about 0.00001, 0.00005, 0.0001, 0.0005, 0.001, 0.005, 0.01, 0.05, 0.1, 0.5 weight percent, based on the total weight of the composition). The composition may provide from about 20% to about 300% (e.g., at least/no more than about 20, 50, 100, 150, 200, 250, 300%) of the recommended daily allowance of niacin.

In some embodiments, the composition includes vitamin D. The primary role of vitamin D is to maintain homeostasis of calcium and phosphorus to maintain bone formation and maintenance, neuromuscular function, and other cellular processes. Vitamin D is beneficial for people as it increases the synthesis of muscle proteins, the concentration of adenosine triphosphate (ATP), strength, jump height, jumping speed and power, as well as the capacity to perform aerobic and anaerobic exercise.

In some embodiments, the composition includes vitamin E. Exercise can cause damage to active muscles. Increased release of muscular enzymes into the plasma and substantial impairment in maximal torque production. The practical implications of this damage have been reviewed and include decreased joint range of motion, increased fatigability, decreased shortening velocity and prolonged strength loss. A 20-30% loss in torque production of the knee extensors has been reported following endurance running. Damage to skeletal muscle cell membranes by reactive oxygen species (ROS), specifically lipid peroxidation, can impair cell viability, leading to necrosis and an acute-phase inflammatory response. Protection from ROS by antioxidants (such as vitamin E) can abrogate muscle damage caused by exercise. Endurance or damaging exercise elicits a stress response analogous to the acute phase immune response. A local response to a stressor such as tissue injury or ROS stimulates production of a group of low molecular weight regulatory proteins, called cytokines, that regulate the inflammatory cascade. ROS may stimulate cytokine production at the level of the skeletal muscle in response to exercise and that vitamin E supplementation may attenuate this stress response.

The term “amino acid” refers to naturally occurring and non-naturally occurring amino acids, as well as amino acid analogs and amino acid mimetics that function in a manner similar to the naturally occurring amino acids. The term “amino acid” further includes, without limitation, precursors, structurally similar compounds, metabolites, salts, esters, and/or isomeric forms of amino acids. Naturally occurring amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs include compounds that have the same basic chemical structure as a naturally occurring amino acid, i.e., a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, the amino acid is theanine, an amino acid analog f the proteinogenic amino acids L-glutamate and L-glutamine.

In addition to being incorporated into proteins, amino acids play numerous roles in physiological processes and supplementation with amino acids has been studied in numerous contexts. For example, phenylalanine is an essential amino acid and plays a key role in the biosynthesis of other amino acids and some neurotransmitters. Similarly, tyrosine is required for the synthesis of adrenal hormones epinephrine, norepinephrine, dopamine, and the thyroid hormones, including thyroxine. Tyrosine, through its effect on neurotransmitters, is used to treat conditions including mood enhancement, appetite suppression, and growth hormone (HGH) stimulation. Tyrosine is also involved in the production of melanin, the pigment responsible for hair and skin color.

The term “taurine” refers to 2-aminoethanesulfonic acid. Taurine is an organic compound that is widely distributed in animal tissues. Taurine is believed to have several biological roles, such as conjugation of bile acids, antioxidation, osmoregulation, membrane stabilization, and modulation of calcium signaling. In one embodiment, taurine is present in an amount from about 0.1% to about 2% by weight (or from about 0.001 g/ml to about 0.02 g/ml). Thus, the composition can include at least about (or no more than about) 0.001, 0.002, 0.003, 0.004, 0.005, 0.006, 0.007, 0.008, 0.009, 0.01, 0.011, 0.012, 0.013, 0.014, 0.15, 0.016, 0.017, 0.018, 0.019, 0.02 g/ml) The term “creatinine” refers to 2-Amino-1-methyl-1H-imidazol-4-ol, a breakdown product of creatine phosphate in muscle.

In some embodiments, the disclosed composition can include xanthine derivatives such as (but are not limited to) caffeine, theobromine, aminophylline, theophylline, paraxanthine and includes precursors, structurally similar compounds, analogs and metabolites of methylated xanthines. The term “caffeine” refers to 1, 3, 7-Trimethylpurine-2,6-dione, a central nervous system (CNS) stimulant of the methylxanthine class. It is mainly used recreationally as a cognitive enhancer, increasing alertness and attentional performance. Caffeine acts by blocking binding of adenosine to the adenosine A1 receptor, which enhances release of the neurotransmitter acetylcholine. Caffeine has a three-dimensional structure similar to that of adenosine, which allows it to bind and block its receptors. Caffeine also increases cyclic AMP levels through nonselective inhibition of phosphodiesterase. In some embodiments, the caffeine can be present in the disclosed composition an amount from about 0.1% to about 8 weight percent, based on the total weight of the composition (e.g., at least/no more than about 0.1, 0.5, 1, 1.5, 2, 2.5, 3, 3.5, 4, 4.5, 5, 5.5, 6, 6.5, 7, 7.5, or 8 weight percent).

Theobromine (also known as xantheose and 3,7-dihydro-3,7-dimethyl-1H-purine-2,6-dione) is the principal alkaloid of the cacao plant. Aminophylline is a compound of the bronchodilator theophylline with ethylenediamine in 2:1 ratio. Theophylline (also known as 1,3-dimethylxanthine) is a phosphodiesterase inhibiting drug used in therapy for respiratory diseases such as chronic obstructive pulmonary disease (COPD) and asthma under a variety of brand names. As a member of the xanthine family, it bears structural and pharmacological similarity to theobromine and caffeine, and is readily found in nature, being present in tea (Camellia sinensis) and cocoa (Theobroma cacao). Paraxanthine refers to 1,7-dimethylxanthine, a dimethyl derivative of xanthine, structurally related to caffeine. In one embodiment, the xanthine derivative(s) is present in an amount from about 0.001% to about 5% by weight (i.e., w/w) (or for some liquid forms from about 0.0001 g/ml to about 0.05 g/ml).

The term “melatonin” refers to N-acetyl-5-methoxy-tryptamine, a natural product found in plants and animals. Melatonin is primarily known in animals as a hormone released by the pineal gland in the brain at night and has long been associated with control of the sleep-wake cycle. Melatonin is effective for treating sleep-wake cycle disturbances in children and adolescents with mental retardation, autism, and other central nervous system disorders. While not bound by any theory, melatonin appears to decrease the time to fall asleep in children with developmental disabilities, such as cerebral palsy, autism, and mental retardation. It may also improve secondary insomnia associated with various sleep-wake cycle disturbances. Supplementation with melatonin is implicated in for insomnia and improving sleep. In one embodiment, melatonin is present in the disclosed composition in an amount from about 0.1% to about 2% by weight (or from about 0.001 g/ml to about 0.02 g/ml). Thus, the melatonin can be present in an amount of at least about (or no more than about) 0.001, 0.002, 0.003, 0.004, 0.005, 0.006, 0.007, 0.008, 0.009, 0.01, 0.011, 0.012, 0.013, 0.014, 0.015, 0.016, 0.017, 0.018, 0.019, or 0.02 g/ml).

The term “choline” refers to 2-Hydroxy-N,N,N-trimethylethan-1-aminium, and is used in the synthesis of phospholipids and, phosphatidylcholine structural components of all cell membranes. Choline is also involved in cell signaling. Choline is also a precursor for acetylcholine, an important neurotransmitter involved in muscle control, memory and mood. Supplementation with choline is implicated in improved cognition, memory and learning and may provide neuroprotective benefits. Choline plays a role in creatine synthesis, creatine-boosting mechanism might be one additional way wherein choline might delay fatigue during marathon-type efforts. The choline derivatives, including precursors, structurally similar compounds, analogs and metabolites of choline may include (but are not limited to) choline, citicholine, phosphocholine, and betaine and phosphatidyl choline. In some embodiments, the choline derivative is citicoline. In one embodiment, the choline derivative is present in an amount from about 0.001% to about 0.1% by weight or from about 0.00001 g/ml to about 0.1 g/ml.

In some embodiments, the disclosed composition can comprise lutein, a xanthophyll and one of 600 known naturally occurring carotenoids. Lutein is synthesized only by plants, and like other xanthophylls is found in high quantities in green leafy vegetables such as spinach, kale and yellow carrots.

In further embodiments, the muscle recovery composition includes additional components that may reduce fatigue and provide energy. Such additional components may include (but are not limited to) one or more of glucuronolactone, glucono delta-lactone, and glucuronic acid. In one embodiment, glucuronolactone derivatives are present in an amount from about 0.1% to about 2% (or from about 0.001 g/ml to about 0.02 g/ml), such as at least about (or no more than about) 0.001, 0.002, 0.003, 0.004, 0.005, 0.006, 0.007, 0.008, 0.009, 0.01, 0.011, 0.012, 0.013, 0.014, 0.15, 0.016, 0.017, 0.018, 0.019, 0.02 g/ml). The composition may also include malic and/or citric acid. Citric and malic acid are intermediates in the conversion of food to energy (e.g., they are part of the citric acid cycle).

In one embodiment, the pH of the composition is from about 1.0 to about 12.0 (e.g., at least/no more than about 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12). In a preferred embodiment, the pH of the composition is acidic. For example, the pH may be from about 6.0 to about 9.0.

In preferred embodiments, the muscle recovery composition includes skin permeation enhancers, such as alcohols and polyols, lactams and their analogs, esters and ethers, surfactants and terpenes, steroids, and fatty acids. The term “alcohol” refers to an organic compound that carries at least one hydroxyl (—OH) functional group bound to a saturated carbon atom. The term “polyol” refers an organic compound containing multiple hydroxyl groups (—OH). The term “lactam” refers to a cyclic amide, formally derived from an amino alkanoic acid. The term “ester” refers to a compound derived from an oxoacid (organic or inorganic) in which at least one hydroxyl group (—OH) is replaced by an alkoxy group (—O—R), as in the substitution reaction of a carboxylic acid and an alcohol. The term “ether” refers to a class of compounds that include an ether group (an oxygen atom connected to two alkyl or aryl groups) and have the general formula R—O—R′, where R and R′ represent the alkyl or aryl groups. The term “surfactant” refers to any chemical compound that decreases the surface tension between two liquids, between a gas and a liquid, or interfacial tension between a liquid and a solid. The term “terpene” refers to a class of natural products comprising compounds with the formula (C5H8)n for n>1. The term “steroid” refers to a biologically active organic compound with four rings arranged in a specific molecular configuration. The term “fatty acid” refers to a carboxylic acid with an aliphatic chain, which is either saturated or unsaturated.

In one embodiment, the composition includes one or more alcohols and/or Polyols in an amount of about 2% w/w to 25% w/w (e.g., at least/no more than about 2, 5,0 10, 15, 20, or 25 weight percent based on the total weight of the composition). In some embodiments, the alcohol is selected from ethanol and/or isopropyl alcohol. Examples include propylene glycol, Azone (laurocapram), Transcutol® P (Tc), oleic acid, ethanol, Polysorbate 80 (Tween 80), Brij 35 (polyoxyethylene lauryl ether) sodium lauryl sulfate (SLS) and N-methyl-pyrrolidone (NMP), urea, dimethylacetamide, dimethylformamide, pyrrolidones.

In some embodiments, the muscle recovery composition includes Dimethyl Sulfoxide (DMSO) skin permeation enhancers is present in an amount 2% w/w to 47% w/w (e.g., at least/no more than about 2, 5, 10, 15, 20, 25, 30, 35, 40, 45, or 47 weight percent).

In some embodiments, preservative agents may be added to the composition. Ethylene diamine tetraacetic acid (“EDTA”) may also be included to improve stability. In one embodiment, EDTA is present in an amount from about 0.002% to about 0.009% (or about 0.00002 g/ml to about 0.00009 g/ml), such as at least about (or no more than about) 0.002, 0.003, 0.004, 0.005, 0.006, 0.007, 0.008, or 0.009 weight percent). In another embodiments, the EDTA is present in an amount from about 0.003% to about 0.007% (or about 0.00003 g/ml to about 0.000007 g/ml). In still another embodiment, the EDTA is present in an amount from about 0.004% to about 0.006% (or about 0.00004 g/ml to about 0.00006 g/ml). In still another embodiment, the EDTA is present in an amount from about 0.002% to about 0.003% (or about 0.00002 g/ml to about 0.00003 g/ml).

The muscle recovery composition may further include at least one preservative. In one embodiment, the preservative is a natural preservative. Embodiments of useful preservatives include, but are not limited to, methyl paraben, propyl paraben, benzoic acid and benzoic acid derivatives such as sodium benzoate, calcium benzoate, potassium benzoate, magnesium benzoate, and combinations thereof. The preservative may include sorbic acid derivatives, such as potassium sorbate. In one embodiment, the preservative is present in an amount from about 0.01% to about 1.0% (or from about 0.0001 g/ml to about 0.01 g/ml). In one embodiment, the preservative is present in an amount from about 0.1% to about 0.8%. Thus, the preservative may be present in the composition in an amount of from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 weight percent, based on the total weight of the composition.

In some embodiments, the composition provides energy, i.e., provides calories. In some embodiments, the composition provides a relatively small amount of food energy. In preferred embodiments, the composition does not contain sugars such as glucose, lactose, sucrose, and/or fructose.

In some embodiments, the muscle recover composition liquid is inserted into an adhesive bandage pod (Appod™) for the desired effects.

In some embodiments, the Appod™ may hold a liquid volume from about 0.25 mls to about 15 mls.

In some embodiments, the Appod™ may hold a liquid volume from about 0.25 mls to about 15 mis from 0.25 hours to 72 hours. Thus, the volume can be at least/no more than about 0.25, 0.5, 0.75, 1, 2, 3, 4, 5, 6, 7, 8, 9, 10, 11, 12, 13, 14, or 15 mLs and the time can be at least/no more than about 0.25, 0.5, 0.75, 1, 3, 6, 9, 12, 15, 18, 21, 24, 27, 30, 33, 36, 39, 41, 44, 47, 50, 53, 56, 59, 62, 65, 68, or 72 hours.

In some embodiments, the Appod™ is inert, hypoallergenic and can be adhered to the skin of humans and animals.

In some embodiments, the Appod™ will be adhered or placed on skin and liquid is added into the Appod™ and it can be refilled more than one time.

In some embodiments, the Appod™ will be adhered or placed on skin and liquid will be prefilled for one time administration.

In some embodiments, the Appod™ may have a silicone or similar inert elastomers as a component that is non-reactive to the liquids, through which the liquid is added into the Appod™.

In some embodiments, the Appod™ can be easily administered by self, itis flexible and applied to knee and other joints and remain adheres for minimum of 0.3 hours to 72 hours (at least/no more than about 0.3, 0.5, 0.75, 1, 3, 6, 9, 12, 15, 18, 21, 24, 27, 30, 33, 36, 39, 41, 44, 47, 50, 53, 56, 59, 62, 65, 68, or 72 hours.

In some embodiments, the Appod™ can be waterproof, during shower and will not soil the cloths and bed.

In some embodiments, the Appod™ is breathable, and provided unidirectional flow the liquid transport to the skin.

As shown in FIG. 1, top face 101 of polymer film includes any suitable material that is breathable and/or provides a wicking layer, such as a polymer. The synthetic polymers that can be cross-linked with natural polymers include (but are not limited to) poly(vinyl alcohol) (PVA), poly(lactic-co-glycolic acid) (PLGA), polylactide (PLA), polyglycolic acid (PGA), polyurethanes (Pus), polyethylene, Polyvinyl chloride (PVC), Polypropylene (PP) poly(ethylene oxide) (PEO)/poly(ethylene glycol) (PEG), poly(hydroxyethyl methacrylate) (PHEMA), and poly(vinyl pyrrolidone) (PVP) polyethylene terephthalate (PET), semi-crystalline polyamides, polyimides such as Nylon 6 or Nylon 66, polyolefins such as polyethylene and polypropylene, polymethylpentene, polyisobutylene, polyolefin copolymers, polyester copolymers, fluoropolymers, poly vinyl acetate, poly vinyl alcohol, polyethylene oxide, functionalized polyolefins, ethylene vinyl acetate copolymers, polytetrafluoroethylene, or amorphous thermoplastic materials to include polycarbonates, polyacrylics, polymethacrylics, polybutadiene, polyisoprene, polychloroprene, random and block copolymers of styrene and dienes, butyl rubber, ethylene-propylene-diene monomer rubber, natural rubber, ethylene-propylene rubber, and mixtures thereof. Other examples of suitable materials may include polyvinyl chloride, thermoplastic polyurethanes, aromatic epoxies, amorphous polyesters, amorphous polyamides, acrylonitrile-butadiene-styrene (ABS) copolymers, polyphenylene oxide alloys, high impact polystyrene, polystyrene copolymers, fluorinated elastomers, polydimethyl siloxane, polyetherimides, methacrylic acid-polyethylene copolymers, impact-modified polyolefins, and/or amorphous fluoropolymers.

In an embodiment as mentioned above, an Appod™ may have an optional interior pad layer 103 as shown in FIG. 1. In some embodiments, an Appod™ may be provided with pad layer comprising non-woven material(s). The pad can also include dome 102 in which liquid is added. Adhesive area 104 surrounds the Appod™. The term “dome” includes a closed structure with an interior that can house the composition as disclosed herein. Thus, the adhesive layer allows the Appod™ to be attached at a desired location on a user (e.g., the site of a sore muscle). The device can be pre-filled with the disclosed composition, or the composition can be added to the dome after the device is attached to the user. The composition can be dispersed from the dome to the skin. In some embodiments, the composition is dispersed onto the pad layer.

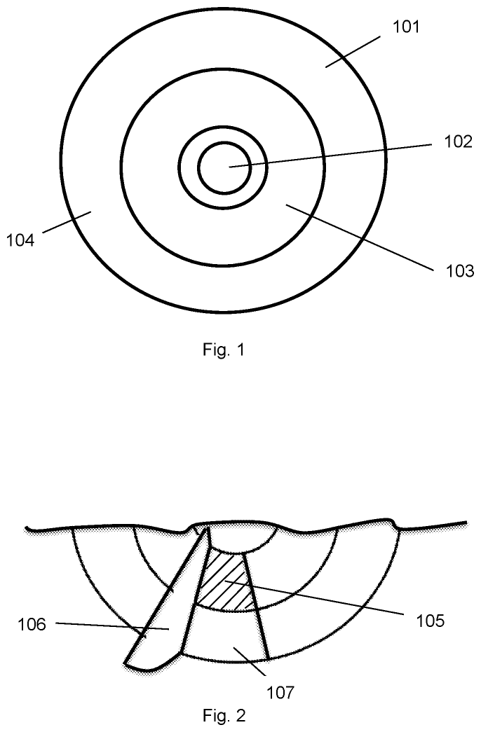

Further, the first adhesive release liner on top of 101 (as shown in FIG. 1) and the second adhesive release paper 106 (as shown in FIG. 2) may be arranged at an outside of the pressurizing area protection portion of the position fixing release liner and are formed to protect a remaining part of the adhesive area of the over patch. Additionally, the first adhesive release liner and the second adhesive release liner may be spaced apart from each other and symmetric to each other with respect to two of the adhesive area protection portions formed at the position fixing release liner. Further, a connection portion of the adhesive area protection area with the pressurizing area protection portion may be formed to have a curved cut line, and the first adhesive release liner and the second adhesive release liner may be formed to be separably cuttable from the adhesive area protection portion along the curved cut line. Additionally, the release liner may be formed to be separably cuttable into a first separation release liner and a second separation release liner, and the first separation release liner and the second separation release liner may be formed to protect a part of the pressurizing area and a part of the adhesive area, respectively, and may be formed to protect a whole area of the adhesive side of the over adhesive bandage pod by dividing the whole area of the adhesive side of the over adhesive bandage pod into two along a mutual cut line.

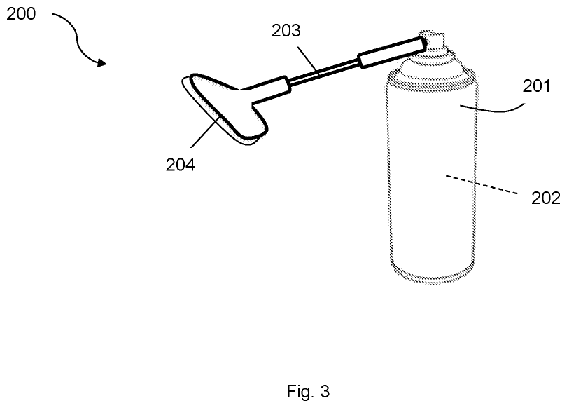

FIG. 3 illustrates an applicator 200 comprising vessel 201 that holds a composition 202 as disclosed herein. Vessel can include any container that can house the composition (e.g., jars, tubes, cans, and the like. Tubing 203 connects the vessel to sponge tip applicator 204. In this way, the composition can be added to the Appod™. For example, the sponge tip can apply the composition to the dome. The sponge tip can apply the composition to the skin.

The Appod™ can be called as applicator device and/or adhesive bandage pod and/or application bandage. In some embodiments, the Appod™ holds the liquid or semi solid or suspension or emulsion onto the particular area of the skin. FIG. 1, illustrates dome 102 into which liquid (the disclosed composition) is added. The dome can be configured in any shape, such as circular or oblong or rectangular or square or triangle or irregular shape. The dome is inert and non-reactive. In some embodiments, the dome is constructed from silicone and/or other elastomers and/or polymers. In some embodiments, the dome is flexible. In some embodiments, dome 102 can be placed at the periphery or close to outer circumference or middle on in an area of the Appod™. The dome can hold 0.25 mL to 25 mL of liquid or semi-solid (e.g., at least/no more than about 0.25, 0.5, 0.75, 1, 2, 3, 4, 5, 6, 7, 8, 9, 10, 11, 12, 13, 14, 15, 16, 17, 18, 19, or 20 mL).

In some embodiment, the Appod™ can adhere on the skin for 0.1 hours to 72 hours (at least/no more than about 0.1, 0.5, 2, 5, 10, 15, 20, 25, 30, 35, 40, 45, 50, 55, 60, 65, 70, or 72 hours) and it allows a user to refill the liquid for more than one time or multiple times while the Appod™ is adhered on the skin

In some embodiments, the Appod™ can hold the liquid longer in the covered environment than the liquid in open surface of the skin.

In some embodiments, the top face 101 of the polymer layer is transparent, allowing consumers to visually look at non-woven layer 101 for the wetness and decide to refill the liquid.

In an embodiment, top face 101 of the polymer layer can have an inlet spout without the requirement of dome 102.

In an embodiment, the top layer can be removed and added a pre-filled liquid container that has pores to drip the liquid in a pre-determined flow rate and reclosed again.

In an embodiment, interior pad layer 103 is a nonwoven or woven fabric made up of gauze, absorbent and/or a nonabsorbent material beneath top face 101 of polymer polyurethane film, that absorbs the added liquid and spreads throughout the area of the skin. In another embodiment, dome 102 holds the added liquid for extended period of time. In another embodiment, the Appod™ can be without interior pad layer 103 (105 is the bottom face of pad layer 103) fabric.

In an embodiment, the Appod™ in FIG. 1 includes adhesive area 104, beginning after padded layer 103 and continues to the periphery of polymer layer 101. As a result, the Appod™ adhered o to the skin on its boundary and the padded layer 103 and silicone dome 102 area will not have the adhesive.

The adhesive bandage pod of the present invention may be provided with an interior pad layer 103 and bottom view 105 in the present invention is a layer provided to have functions of spreading and holding the liquid added into the adhesive pod for prolong or extended time delivery into the skin, protecting the affected part Usually, gauze, nonwoven fabric, woven fabric, knitted fabric, compressed fiber, hydrophilic colloid, or other absorbent material like cotton and non-absorbent material like viscose, rayon, nylon etc. can be used. In addition, the pad layer 103106
may hold a disinfectant, a therapeutic agent, emollient, and/or other agents as needed. The size of the pad layer usually in the range of 5% to 90%, such as 10% to 70% or 15% to 60% when the area of the entire bandage adhesive pod 100%.

In some embodiments, polymer layer top face 101 is very flexible, stretchable, and elongates and prevents breaking. The top layer can be highly porous and high moisture vapor transmission rate. The top layer can include polyurethane. Further, it can have a top release liner. In some embodiments, layer 101 has a thickness of 2 μm to 1.0 cms, In a refinement, the thickness of the fabric layer is about 3 micron to about 70 mm. In a refinement, the thickness of the fabric is present in an amount from about 5 microns to about 1000 microns. In still another refinement, the thickness of the fabric layer is present in an amount from about 10 μm to about 700 μm.

In some embodiments, the top layer release liner is removed after the Appod™ is applied on the skin.

In some embodiment, the top release liner provides the structural integrity of the polymer film top face 101.

In some embodiments, the thickness of the of the polymer layer is about 3 mm to 10 mm (e.g., at least/no more than about 3, 4, 5, 6, 7, 8, 9, 10 mm), such as from 10 mm to 300 mm.

In some embodiments, the top layer polymer for the Appod™ can include ultrafine urethane nonwoven fabric in the range of about 5 μm to 50 μm (at least/no more than about 5, 10, 15, 20, 25, 30, 35, 40, 45, or 50 um), such as 10 μm to 25 μm.

In some embodiments, top face 101 of the polymer layer on the basis weight of the ultrafine urethane non-woven fabric is about 30 to 320 g/m² (e.g., at least/no more than about 30, 50, 100, 150, 200, 250, 300, 320 g/m2). Thus, the basis weight can be about 50 to 150 g/m² or 60 to 120 g/m².

In some embodiments, the polymer layer top face 101 can be multiple layered. The top layer can include visibly colorful designs. The top layer can be multiple layered of different polymers to hold the liquid or to provide occlusive properties.

In some embodiments the polymer layer has adhesive on the bottom as shown in element 107. The adhesive can be fully covered for the Appod™.

In some embodiments, the adhesive is covered on the periphery or outer part in a range of 0.1 cm to 5 cm (at least/no more than about 0.1, 0.5, 1, 1.5, 2, 2.5, 3, 3.5, 4, 4.5, or 5 cm) from its outer end, such as 1 cm to 3 cm of adhesive area.

In some embodiments, adhesive layer 107 includes adhesive that may be placed over on-skin unit before or after removal of release liner 106. In an embodiment, providing a release liner that does not cover or encircle interior dome 102 allows Appod™ to be placed over an on-skin unit and for subsequent removal of release liner 106, thus reducing the extent to which the adhesive of Appod™ is disturbed by the user or impacted by environmental conditions (dirt, debris, water, etc.). After the release liner is removed, the bottom layer of the device includes adhesive 107. In some embodiments, bottom face 105 of the pad layer 103 may have adhesive that can adhere to the skin. In some embodiments, the bottom face 105 the pad layer lacks an adhesive.

In other embodiments, release liner 106 may be provided with more or less parts that may or may not overlap. In an embodiment, tabs may or may not be provided, and, if provided, such tabs may be of any suitable size, shape, color, etc. In an embodiment, tabs may be color coded and/or numbered to indicate the recommended order of removing the various release liner parts.

The present invention can include a release liner 106 in the bottom and a release liner on top face 101 of the polymer layer. The release liner can be laminated on the surface of the breathable pressure-sensitive adhesive layer which can comprise ultrafine urethane nonwoven fabric. The release liner may be one conventionally used in the field adhesive bandages, for example, a paper substrate such as a woodfree paper or a cellophane coated with a release agent and subjected to a release treatment, a polyester film, or the like. The release liner may be a release sheet having a size and shape such that it covers the entire adhesive bandage pod on top and bottom or may be a release liner having an area larger than the adhesive bandage pod. The release liner may be a release sheet divided into 2 or more sheets, and the fold-over portion may be provided in at least 1 sheet of the release liner divided into 2 or more sheets. Further, the adhesive bandage pod may be divided into 3 or more pieces of release liner sheets by disposing 1 or more pieces of release liner 106 covering a part of the pad layer bottom face 105 and disposing 2 or more pieces of release liner on the adhesive layer having air permeability and the adhesive sheet around the pad layer bottom face 105.

In other embodiments, adhesive 107 can be an acrylic adhesive, a rubber adhesive, a silicone adhesive, or combinations thereof. An adhesive with little skin irritation can be used.

Examples of the rubber-based adhesive that can be used include a composition obtained by blending a tackifier resin, a softening agent, and the like with a rubber base such as a synthetic polyisoprene rubber or a styrene-isoprene-styrene block copolymer.

Examples of the acrylic pressure-sensitive adhesive include homopolymers or copolymers of long-chain (meth) acrylate monomers having about 4 to 12 carbon atoms such as butyl acrylate, 2-ethylhexyl acrylate and isononyl acrylate, and copolymers obtained by copolymerizing 2 to 70 wt % or more of other copolymerizable monomers such as (meth) acrylic acid, vinyl acetate, styrene, vinylpyrrolidone, acrylamide, hydroxyethyl acrylate and hydroxypropyl acrylate with the (meth) acrylate monomers as the main component. The acrylic pressure-sensitive adhesive may be a solvent-type pressure-sensitive adhesive obtained by polymerizing the monomer in an organic solvent such as toluene, hexane, or ethyl acetate in a nitrogen atmosphere using a peroxide such as benzoyl peroxide as an initiator or may be an emulsion-type pressure-sensitive adhesive obtained by emulsifying and dispersing the monomer in water with an emulsifier and then polymerizing the monomer. After polymerization, a proper amount of a polyfunctional resin such as an epoxy resin is added before coating to the sheet or film substrate, thereby crosslinking the polymer.

In other embodiments adhesive layer 107 of the adhesive bandage pod (Appod™) of the present invention is made of the adhesive layer having air permeability, so that the adhesive layer and the adhesive bandage have good air permeability and cushioning properties and have less irritation to skin properties and good adhesion to the skin.

In other embodiments, the adhesive layer 107 of the adhesive bandage pod (Appod™) of the present invention can have air permeability in between 1 seconds/100 mL to 30 seconds/100 mL. The adhesive air permeability is tested by using Gurley air permeability apparatus according to JIS-P8117 The air permeability measured by a TESTER (TESTER INDUSTRIAL CO., LTD.) was 20 seconds/100 mL or less. The air permeability of the pressure-sensitive adhesive layer can be about 10 seconds/100 mL or less, such as about 5 seconds/100 mL or less, and such as about 1 second/100 mL or less.

The disclosed muscle recovery composition liquid can include a topical Hands free Applicator Drug delivery System (THADS) for individual or for multimodal method of drug cosmetic, natural supplement treatment, includes an airless metered pump product container or a suitable container with a modified nozzle to hold the applicator wand/tube that is intended for unit dose or multiple dose application. The device holds topical liquid in “ready to act state” in which a product outlet can connect and disconnect the one end of a wand of the applicator, the wand is straight or flexible/bendable, is 0.01 inch to 30 inch in length (e.g., at least/no more than about 0.01, 0.05, 1, 5, 10, 15, 20, 25, or 30 inches) that directs drug product to another end which is connected to a applicator sponge tip. The sponge tip can be connected or disconnected, the applicator sponge tip includes soft sponge which holds the drug product, and the wand can be disconnected from a first end after pumping the drug product, and drug is applied to the skin in the area of treatment, with little pressure or rubbing. The drug will be released from the sponge tip or being applied to the skin as “Hand free approach”. When the soft sponge tip of the applicator second end is disconnected, the wand tube can be inserted into the opening of an adhesive bandage pod (Appod™).

Tables 1-6 provide a set of components that may be introduced into such a liquid. The amounts provided in Tables 1-6 are particularly useful to form compositions mg/mL.

TABLE 1

| Ingredients | mg |
|---|---|
| Menthol | 10-200 |
| Camphor | 5-160 |
| Methyl Salicylate | 25-400 |
| Propylene Glycol | 10-270 |
| Ethanol | 50-200 |
| Purified water USP | 25-800 |
| Glycerin | 2-50 |
| Vitamin D | 0-50 |
| Vitamin B6 | 10-60 |
| Caffeine | 10-225 |
| Niacinamide | 0-60 |
| Sodium chloride | 0.01-5 |
| Magnesium Chloride | 0.01 2 |
| Strontium Nitrate | 0.1-5 |
| Dimethyl sulfoxide | 100-400 |

The liquid composition of Table 1 when kept in an open beaker, the liquid became cloudy and white in color, whereas when the same liquid is kept in airtight bottle, it remains clear. In some embodiments, the composition will be used an airtight container as a unit dose or in an Airless container or airless bottle or airless pump bottle.

TABLE 2

| Ingredients | mg |
|---|---|
| Menthol | 10-200 |
| Camphor | 5-160 |
| Methyl Salicylate | 0-400 |
| Propylene Glycol | 10-270 |
| Ethanol | 50-200 |
| Purified water USP | 25-800 |
| Glycerin | 2-50 |
| Vitamin D | 0-50 |
| Melatonin | 0.5-5 |
| Caffeine | 0-200 |
| Niacinamide | 0-60 |
| Sodium chloride | 0.01-5 |
| Magnesium Chloride | 0.02-2 |
| Strontium Nitrate | 0.1-5 |
| Dimethyl sulfoxide | 100-400 |

TABLE 3

| Ingredients | mg |
|---|---|
| Menthol | 10-200 |
| Camphor | 5-260 |
| Propylene Glycol | 10-270 |
| Ethanol | 50-200 |
| Purified water USP | 25-800 |
| Glycerin | 2-50 |
| Vitamin D | 0-50 |
| Vitamin B6 | 10-60 |
| Caffeine | 10-225 |
| L-Alanine | 0-60 |
| Sodium chloride | 0.01-5 |
| Strontium Nitrate | 0.1-5 |
| Dimethyl sulfoxide | 100-400 |
| L-Alanine | 0-10 |
| L-Glutamine | 0-10 |

TABLE 4

| Ingredients | mg |
|---|---|
| Menthol | 10-400 |
| Propylene Glycol | 10-270 |
| Ethanol | 50-200 |
| Purified water USP | 25-800 |
| Glycerin | 2-50 |
| Vitamin D | 0-50 |
| Melatonin | 0.5-5 |
| Caffeine | 0-200 |
| Niacinamide | 0-60 |
| Sodium chloride | 0.01-5 |
| Magnesium Chloride | 0.02-2 |
| Strontium Nitrate | 0.1-5 |
| Dimethyl sulfoxide | 100-400 |

TABLE 5

| Ingredients | mg |
|---|---|
| Menthol | 10-200 |
| Lidocaine | 10 to 150 |
| Propylene Glycol | 10-270 |
| Ethanol | 50-200 |
| Purified water USP | 25-800 |
| Glycerin | 2-50 |
| Vitamin D | 0-50 |
| Vitamin B6 | 10-60 |
| Caffeine | 10-225 |
| L-Alanine | 0-60 |
| Sodium chloride | 0.01-5 |
| Strontium Nitrate | 0.1-5 |
| Dimethyl sulfoxide | 100-400 |
| L-Alanine | 0-10 |
| L-Glutamine | 0-10 |

TABLE 6

| Ingredients | mg |
|---|---|
| Menthol | 10-200 |
| Camphor | 5-160 |
| Methyl Salicylate | 25-400 |
| Propylene Glycol | 10-270 |
| Ethanol | 50-200 |
| Purified water USP | 25-800 |
| Glycerin | 2-50 |
| L-Theanine | 0.01-5 |
| Niacinamide | 0-60 |
| Sodium chloride | 0.01-5 |
| Melatonin | 0.5-5 |
| Strontium Nitrate | 0.1-5 |
| Dimethyl sulfoxide | 100-400 |

It is to be understood that this disclosure is not limited to the embodiments described above as specific components and conditions may vary. Furthermore, the terminology used herein is used only for the purpose of describing particular embodiments and is not intended to be limiting in any way.

It is an object of the present invention to provide a topical delivery system and for an muscle recovery composition which avoids the draw backs known from the prior art. It is a further object of the present invention to provide a technology/composition which is able to administrate the topical delivery hands free and delivery drugs, very efficaciously with high consumer compliance to a subject over a period of time in a consistent and controllable way.

Although certain embodiments have been illustrated and described herein for purposes of description of preferred embodiments, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

While the present disclosure has been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

Any discussion of references cited in this description of the prior art merely summarizes the disclosures of the cited references no admission is made that any cited reference or portion thereof is relevant prior art. Applicant reserves the right to challenge the accuracy, relevancy and veracity of the cited references.

### REFERENCES

Driskell et al, Sports Nutrition: Vitamins and Trace Elements, Second Edition. 2006, pages, 69, 82-84, 148

What is claimed is:

1. a topical workout muscle recovery composition comprising:
a. pain reliever;
b. Water;
c. a nonsteroidal anti-inflammatory drug derivative selected from the group comprising diclofenac; ibuprofen; piroxicam; ketorolac; a local anesthetic drug derivative selected from the group comprising lidocaine, benzocaine; a rubefacient/counter irritant drug derivative selected from the group comprising methyl salicylate, trolamine salicylate, nicotinate esters, capsaicin derived from chili pepper, Capsicum minimum, menthol, minoxidil, camphor and thurfyl nicotinate; and naturally occurring plant derivatives selected from the group comprising Eucalyptus, clove, Arnica montana, Boswellia carterii, Calendula officinalis and Vamellia dinensis; and combinations thereof,
d. at least one inorganic salt derived from Sodium, Magnesium, potassium, copper, strontium, zinc, or combinations thereof;
e. an amino acid or a dipeptide and its salt selected from at least one member selected from the group comprising leucine, isoleucine, valine, glutamine, creatine, carnitine, bxetaine, beta-Alanine, taurine, and combinations thereof;
f. a natural supplement selected from the group comprising melatonin, caffeine, L-theanine, ashwagandha, Ginseng, biotin, keratin, gamma ammino butyric acid, vitamin A, vitamin D, vitamin C, vitamin B complex, vitamin B12, and coenzyme Q10 (CoQ10), or combinations thereof
g. at least one preservative;
wherein said composition is in liquid to semisolid in nature or consistency in an airless bottle.

2. A topical workout muscle recovery composition comprising:
a. a pain reliever;
b. Water;
c. a nonsteroidal anti-inflammatory drug derivative selected from the group comprising diclofenac; ibuprofen; piroxicam; ketorolac; a local anesthetic drug derivative selected from lidocaine, Benzocaine; a rubefacient/counter irritant drug derivative selected from methyl salicylate or trolamine salicylate; nicotinate esters; capsaicin, derived from chili pepper; Capsicum minimum; menthol; minoxidil; camphor, thurfyl nicotinate; and naturally occurring plant derivatives selected from Eucalyptus, clove, Arnica Montana, Boswellia carterii, Calendula Officinalis and Camellia Sinensis; and combinations thereof;
d. at least one member selected from amino acid or a dipeptides and its salts selected from the group containing Leucine, Isoleucine, valine, Glutamine, creatine, carnitine, bxetaine, beta-Alanine and taurine and combinations thereof;
e. at least one natural supplement selected from melatonin, caffeine, L-theanine, ashwagandha, Ginseng, biotin, keratin, gamma ammino butyric acid, vitamin A, vitamin D, vitamin C, vitamin B complex, vitamin B12, and coenzyme Q10 (CoQ10);
f. at least one preservative;
wherein said composition is in liquid to semisolid in nature or consistency in an airless bottle.

3. A topical workout muscle recovery composition water comprising:
a. pain reliever;
b. a nonsteroidal anti-inflammatory drug derivative selected from the group comprising diclofenac; ibuprofen; piroxicam; ketorolac; a local anesthetic drug derivative selected from the group lidocaine, benzocaine; a rubefacient/counter irritants drug derivative selected from the group methyl salicylate, trolamine salicylate; Nicotinate esters; Capsaicin derived from chili pepper; Capsicum minimum; Menthol; Minoxidil; camphor; Thurfyl nicotinate; and naturally occurring plant derivatives selected from Eucalyptus, clove, Arnica Montana, Boswellia carterii, Calendula Officinalis and Camellia Sinensis; and combinations thereof;
c. at least one inorganic salt derived from Sodium, Magnesium, Potassium, copper, strontium, or zinc;
d. at least one natural supplement selected from Melatonin, caffeine, L-Theanine, Ashwagandha, Ginseng, Biotin, Keratin, gamma ammino butyric acid, Vitamin A, vitamin D, vitamin C, vitamin B complex, vitamin B12, and Coenzyme Q10 (CoQ10);
e. at least one preservative;
wherein said composition is in liquid to semisolid in nature or consistency in an airless bottles.

4. A topical workout muscle recovery composition water comprising:
a. pain reliever;
b. a nonsteroidal anti-inflammatory drug derivative selected from diclofenac; ibuprofen; piroxicam; ketorolac; a local anesthetic drug derivative selected from the group lidocaine, benzocaine; a rubefacient/counter irritants drug derivative selected from the group methyl salicylate and trolamine salicylate; Nicotinate esters; Capsaicin derived from chili pepper; Capsicum minimum; Menthol; Minoxidil; camphor, Thurfyl nicotinate; and naturally occurring plant derivatives selected from Eucalyptus, clove, Arnica Montana, Boswellia carterii, Calendula Officinalis and Camellia Sinensis; and combinations thereof,
c. at least one inorganic salt derived from Sodium, Magnesium, Potassium, copper, strontium, or zinc;
d. at least one member selected from amino acid or a dipeptide and its salts selected from the group containing Leucine, Isoleucine, valine, Glutamine, creatine, carnitine, bxetaine, beta-Alanine and taurine and combinations thereof; wherein said composition is in liquid to semisolid in nature or consistency in an airless bottles.

5. The topical workout muscle recovery composition of claim 1, wherein the composition a liquid, is in a completely dissolved state and “ready to act form” and provides consistent effect as a solution or nanosized or micronized suspension, gel or an emulsion with a viscosity of 2000 cP or less; and wherein the composition further comprises caffeine and collogen protein.

6. The topical workout muscle recovery composition of claim 1, comprising about 1% to 90% by weight of water and a skin permeation enhancer; wherein the composition is mixed with polymers to provide a sustainable action for longer period; and wherein the composition can be applied using an THADS applicator in airless container or an Adhesive bandage pod.

7. The topical workout muscle recovery composition of claim 6, wherein the adhesive bandage pod is configured to be applied on the skin of human or animals, and wherein the adhesive bandage pod holds the liquid or semi-liquid in place, and wherein the adhesive bandage pod is configured to prevent the soiling of cloths and provide hands-free application of a topical product or products.

8. The topical workout muscle recovery composition of claim 6, wherein the adhesive bandage pod is configured to be applied to the skin, wherein the topical solution/product is added into the adhesive bandage pod for local or systemic applications, wherein the drug product is not adhered to adhesive.

9. The topical workout muscle recovery composition of claim 6, wherein the adhesive bandage pod is configured to hold the topical composition in place and prevent any reduction or loss of the composition from any external contact of the treatment via rubbing of a hand or absorption of clothing.

10. The topical workout muscle recovery composition of claim 6 wherein the adhesive bandage pod is configured to provide rapid onset and supply a constant rate of supply of topical products in an enclosed area of skin for an extended period and wherein the topical product can be refilled if needed.

11. The topical workout muscle recovery composition of claim 6, wherein the adhesive bandage pod is configured to administer more than one topical product simultaneously in a same treatment area of skin for multimodal treatment.

12. The topical workout muscle recovery composition of claim 6, wherein the adhesive bandage pod comprises an adhesive, an occlusive or non-occlusive polymer top layer, and a non-woven or woven fabric to hold or to spread the topical product, and a release liner.

13. The topical workout muscle recovery composition of claim 6, wherein the adhesive bandage pod comprises a predetermined closed or a refillable chamber or holder for the topical product that can have predetermined quantities of topical product, wherein the topical product will be released from the chamber or holder at a predetermined rate to an application area, wherein the adhesive bandage pod (Appod™) optinally comprises non-woven or woven fabric.

14. The Topical workout muscle recovery composition of claim 1, wherein said amino acid is glutamine or its derivatives in an amount from about 0.01% to about 20 weight percent, wherein the composition optionally further includes L-Analy-L Glutamine.

15. The Topical workout muscle recovery composition of claim 6, comprising about 0.05 mL-5 ml of water per pound of muscle and wherein the preservative is present in an amount from about 0.015% to about 2 weight % and is from sodium benzoate, potassium sorbate, benzoic acid, sorbic acid, levulinic acid, anisic acid, methylparaben, ethylparaben, propylparaben, butylparaben, isobutylparaben, methylisothiazolinone, imadozolidinyl urea, diazolidinyl urea, phenoxyethanol, caprylyl glycol, EDTA, and combinations thereof.

16. The Topical workout muscle recovery composition of claim 1, wherein said inorganic salt is present in an amount from about 0.1% to about 200% of daily volume.

17. The Topical workout muscle recovery composition of claim 1, wherein said caffeine is present in an amount from about 0.1% to about 40 weight % of the composition.

18. The topical workout muscle recovery composition of claim 6, wherein the adhesive bandage pod comprises a frame having an upper surface, a lower surface, an outer boundary and an inner boundary, wherein said inner boundary defines an interior opening configured to receive a topical dosage product selected from a solution suspension, emulsion ointment, cream, or topical powder.

19. The topical workout muscle recovery composition of claim 18, wherein the adhesive bandage pod comprises an adhesive disposed on said lower surface of said frame; and a release liner removably coupled to said adhesive, wherein the adhesive in the said lower surface frame is continuous or discontinuous, and wherein the adhesive is positioned on all sides or on less than all sides of the frame.

20. The Topical workout muscle recovery composition of claim 1, wherein said amino acid is present in an amount of about 0.1-18 weight percent, and wherein the amino acid is selected from one or more of Glutamine, Alanine, Creatine, leucine, isoleucine, valine, or an amino acid derivative.

21. The topical workout muscle recovery composition of claim 6, wherein the adhesive bandage pod comprises an outer layer material with an opening to add the topical product, a non-woven fabric layer or layers to spread or hold the topical products, wherein said outer layer material is transparent or non-transparent, waterproof, flexible, permeable to water vapor, and resistant to penetration by microorganisms.

22. The topical workout muscle recovery composition of claim 18, wherein the composition is not bound in an adhesive within the wherein the adhesive bandage pod but is dispersed or spread free in an area defined by said interior opening of said frame and very close to the skin.

23. The topical workout muscle recovery composition of claim 6, wherein said release liner of the adhesive bandage pod comprises multiple parts each removably coupled to said adhesive on the lower surface and also on said upper surface.

24. The topical workout muscle recovery composition of claim 6, wherein at least two of said multiple parts of said adhesive bandage pod release liner overlap and comprise tabs for grasping and removing said release liner.

25. The topical workout muscle recovery composition of claim 24, wherein said tabs are at least one of color coded or numbered to indicate a recommended order of removal.

26. The topical workout muscle recovery composition of claim 6, wherein the adhesive bandage pod has a frame having an upper surface, a lower surface, an outer boundary which contains adhesive and an inner boundary which contains a silicone dome, wherein in between inner boundary and outer boundary lies an intermediate layer that comprises non-woven fabric that absorbs the liquid and holds and delivers the product to the skin.
